(19) United States
(12) Patent Application Publication
(10) Pub. No.: US 2025/0186498 A1
(43) Pub. Date: Jun. 12, 2025

(54) COMPOSITION FOR HEARING LOSS MITIGATION AND USE THEREOF

(71) Applicant: TAIWAN MITOCHONDRION APPLIED TECHNOLOGY CO., LTD., Zhubei City, Hsinchu County (TW)

(72) Inventors: Han-Chung CHENG, Zhubei City (TW); Hui-Ching TSENG, Zhubei City (TW); Chih-Kai HSU, Zhubei City (TW); Li-Hsin YAO, Zhubei City (TW); Tsung-You YAO, Zhubei City (TW); Cheng-Wei WU, Zhubei City (TW)

(21) Appl. No.: 18/845,291

(22) PCT Filed: May 16, 2023
(86) PCT No.: PCT/CN2023/094547

§ 371 Date: Sep. 9, 2024

Related U.S. Application Data
(60) Provisional application No. 63/342,568, filed on May 16, 2022.

(51) Int. Cl. A61K 35/19; A61P 27/16; A61P 39/06

(52) U.S. Cl. CPC A61K 35/19; A61P 27/16; A61P 39/06

(57) ABSTRACT

A composition for hearing loss mitigation and use thereof are disclosed. The composition includes mitochondria and a biocompatible carrier. The composition can mitigate, repair, ameliorate, or treat hearing loss and can be expected to be a composition or medicament that is able to mitigate, repair, ameliorate, or hearing loss while having both safety and effectiveness.

### CROSS-REFERENCE TO RELATED APPLICATIONS

This application is a U.S. National Phase of International Application No. PCT/CN2023/094547, filed on May 16, 2023, which claims the benefit of U.S. provisional patent application Ser. No. 63/342,568, filed on May 16, 2022, the entire contents of which are hereby incorporated by reference.

### BACKGROUND

### 1. Technical Field

The present disclosure relates to a composition for hearing loss mitigation and use thereof.

### 2. Related Art

Approximately one billion people worldwide have hearing loss. Hearing loss may be caused by various factors, such as aging, ototoxic drugs, excessive noise, and genetic disorders. Hearing loss may include conductive hearing loss, central hearing loss, sensorineural hearing loss, or mixed hearing loss. Conductive hearing loss originates from the outer or middle ear. Common causes of conductive hearing loss include earwax blockage, tympanic membrane perforation, middle ear effusion, and ossicular chain discontinuity. Conductive hearing loss is typically mild to moderate and may often be improved through surgery. Sensorineural hearing loss originates from the inner ear or auditory nerve. Causes of sensorineural hearing loss include infections from filterable viruses, treatment with ototoxic drugs, aging, or exposure to noisy environments. Sensorineural hearing loss typically involves damage to the inner ear and often features phenomena like recruitment, where loud sounds may be uncomfortable while softer sounds are harder to hear. Individuals with this type of hearing loss usually have worse hearing ability in the high-frequency range compared to the low-frequency range. As a result, they may hear low-frequency vowels more clearly but struggle to understand high-frequency consonants. Clinically, this often results in the ability to hear someone speaking but difficulty understanding the content of the speech. Central hearing loss originates from the central auditory nervous system and may be caused by aging, brain injury, or other neurological disorders. Central hearing loss often results in decreased auditory memory and comprehension abilities. Mixed hearing loss involves the simultaneous presence of two or more types of hearing impairment.

In clinical practice, cisplatin-based anticancer drugs are common ototoxic agents that may cause irreversible, progressive, bilateral, and cumulative hearing loss. Cisplatin-based drugs affect cochlear hair cells and spiral ganglion neurons, leading to apoptosis of cochlear hair cells. Additionally, these drugs may disrupt antioxidant system-related enzymes, such as catalase, glutathione reductase, and superoxide dismutase, resulting in the accumulation of free radicals and damage to cochlear tissue. Currently, there are no effective medications for treating hearing loss. The typical approach to improving hearing loss is through the implantation of a cochlear implant and administration of glucocorticoids to suppress foreign body reactions and enhance the survival rate of cochlear hair cells and spiral ganglion neurons, thereby reducing inflammation and functional decline in the ear.

However, cochlear implantation is an invasive treatment associated with certain risks, and the medications used to suppress foreign body reactions may also have potential side effects. Therefore, there remains an urgent need to develop compositions or medicament that can mitigate or repair hearing loss while having both safety and effectiveness.

### SUMMARY

The present disclosure provides an alternative approach to improving hearing loss other than cochlear implantation, which may avoid the side effects associated with the use of anti-rejection medicament, and reveal the possibility of treating hearing loss with medicament, thus opening up a new direction for the treatment of hearing loss.

In one embodiment of the present disclosure, a use of mitochondria in manufacturing a composition for hearing loss mitigation is provided.

In one embodiment of the present disclosure, a composition includes mitochondria and a biocompatible carrier.

According to the embodiments of the present disclosure, the composition including mitochondria may reduce the damage caused by peroxides to ear cells, thereby reducing the death of ear cells. Also, the composition including mitochondria may reduce the production of reactive oxygen species (ROS) in ear cells induced by peroxides, thereby reducing the further damage caused by the reactive oxygen species to ear cells. In addition, the composition including mitochondria may reduce the damage caused by peroxides to the mitochondria in ear cells, thereby improving the mitochondrial function of ear cells. Further, the composition including mitochondria and extracellular vesicles exhibits synergistic effects in mitigating, repairing, ameliorating, or treating the damage to ear cells, significantly reduces the death of ear cells caused by peroxides, further reduces the production of reactive oxygen species and the associated damage, and further improves the mitochondrial function of ear cells. Therefore, the composition of the embodiments of the present disclosure may achieve the purposes of mitigating, repairing, ameliorating, or treating hearing loss, and may be expected to be a composition or medicament that is able to mitigate, repair, ameliorate or treat hearing loss while having both safety and effectiveness.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 shows the cell viability of the human cochlear hair cells treated with H₂O₂, relative to the control group.

FIG. 2 shows the ROS production of the human cochlear hair cells treated with H₂O₂, relative to the control group.

FIG. 3 shows the flow cytometry analysis of the ROS production of the human cochlear hair cells treated with H₂O₂, relative to the control group.

FIG. 4 shows the ROS production calculated from the flow cytometry analysis of FIG. 3, relative to the control group.

FIG. 5 shows the ratio of JC-1 monomer/JC-1 aggregate (JC-1 ratio) of the mitochondria in the human cochlear hair cells treated with H₂O₂.

FIG. 6 shows the cell viability of the human cochlear hair cells treated with H₂O₂ and then treated with the compositions of the examples and the comparative examples, relative to the control group.

FIG. 7 shows the ROS production of the human cochlear hair cells treated with H₂O₂ and then treated with the compositions of the examples and the comparative examples, relative to the control group.

FIG. 8 shows the ratio of JC-1 monomer/JC-1 aggregate (JC-1 ratio) of the mitochondria in the human cochlear hair cells treated with H₂O₂ and then treated with the compositions of the examples and the comparative examples, relative to the control group.

FIG. 9 shows the ratio of JC-1 monomer/JC-1 aggregate (JC-1 ratio) of the mitochondria in the human cochlear hair cells treated with H₂O₂ and then treated with the compositions of the examples and the comparative examples, relative to the control group.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

In the present disclosure, features and conditions such as values, numbers, contents, amounts or concentrations presented as a range are merely for convenience and brevity. Therefore, a range should be interpreted as encompassing all possible subranges and individual numerals or values therein, including integers and non-integers. For example, a range of “1.0 to 4.0”, “1.0˜4.0” or “between 1.0 and 4.0” should be understood as explicitly disclosing all subranges such as 1.0 to 4.0, 1.0 to 3.0, 1.0 to 2.0, 2.0 to 4.0, 2.0 to 3.0, 3.0 to 4.0 and so on and encompassing the end points of the ranges, particularly, a subrange defined by numerals or values whose significant digits are represented by 0, 1, 2, 3, 4, 5, 6, 7, 8, 9 or 0 should be interpreted as encompassing all significant digits of numerals or values within the range defined by the end points, for example “1.00 to 2.00” should be interpreted as encompassing all individual values such as 1.00, 1.10, 1.20, 1.30, 1.40, 1.50, 1.60, 1.70, 1.80, 1.90 and so on.

Given the intended purposes and advantages of this disclosure are achieved, numerals have the precision of their significant digits. For example, 10.0 should be understood as covering a range of 9.50 to 10.49.

In the embodiment of the present disclosure, a composition includes mitochondria and a biocompatible carrier. The composition according to one embodiment of the present disclosure may act on ear cells to mitigate, repair, ameliorate or treat hearing loss. The ear cells may include cochlear hair cells, cochlear nerve cells, spiral ganglion neurons or vestibular nerve cells. The hearing loss may include conductive hearing loss, central hearing loss, sensorineural hearing loss or mixed hearing loss.

The mitochondria may be taken from any cell having mitochondria, preferably taken from mammalian monocytes or stem cells, but not limited thereto. For example, the stem cells may be adipose-derived mesenchymal stem cells, embryonic stem cells, mesenchymal stem cells, hematopoietic stem cells, CD34+ stem cells, induced pluripotent stem cells or bone marrow stem cells. In some embodiments, the source of the mitochondria depends on a subject to which the composition is administered, and the mitochondria may be preferably taken from cells of the same species as the subject to which the composition is administered. For example, the mitochondria may be taken from human cells when the subject to which the composition is administered is human, while the mitochondria may be taken from dog cells when the subject to which the composition is administered is a dog. In some embodiment, the mitochondria may be taken from cells of species different from the subject to which the composition is administered, or the mitochondria may also be exogenous mitochondria obtained from in vitro preservation or in vitro culture. In some embodiments, the mitochondria may be used directly after being taken out, or may be used after in vitro preservation or in vitro culture.

The biocompatible carrier may maintain the mitochondrial activity, encapsulate the mitochondria, facilitate the mitochondria to enter into cells, or enhance mitochondrial targeting and specificity. The biocompatible carrier may include a pharmaceutically acceptable carrier. The pharmaceutically acceptable carrier may include a carrier used in any standard medical products or cosmetic products. The biocompatible carrier may be a semi-solid or liquid depending on the form of the composition. For example, the biocompatible carrier may include, but not limited to, water, physiological saline or buffer solution.

In the embodiment, the concentration of the mitochondria in the composition may be 40 μg/mL to 200 μg/mL. In another embodiment, the concentration of the mitochondria in the composition may be 40 μg/mL to 60 μg/mL. In other embodiment, the concentration of the mitochondria in the composition may be 60 μg/mL to 160 μg/mL. In other embodiment, the concentration of the mitochondria in the composition may be 160 μg/mL to 200 μg/mL. In other embodiment, the concentration of the mitochondria in the composition may be at least 60 μg/mL.

In the embodiment, the effective dose of the mitochondria in the composition may be 10 μg to 50 μg. In another embodiment, the effective dose of the mitochondria in the composition may be 10 μg to 15 μg. In other embodiment, the effective dose of the mitochondria in the composition may be 15 μg to 40 μg. In other embodiment, the effective dose of the mitochondria in the composition may be 40 μg to 50 μg. In other embodiment, the effective dose of the mitochondria in the composition may be at least 15 μg.

In the embodiment, the composition may be administered to the ear cells in a manner of oral administration, injection, coating, topically applying, or dripping.

In another embodiment, the composition may further include extracellular vesicles. The extracellular vesicles may be derived from platelet-rich plasma (PRP), stem cells monocytes, fibroblasts, neurons, smooth muscle cells, endothelial cells or epidermal cells. Hereinafter, the extracellular vesicles that is derived from platelet-rich plasma will be referred to as PRP-derived extracellular vesicles or PRP-EVs. The manufacturing method of PRP-derived extracellular vesicles will be described in the following embodiments. The PRP-derived extracellular vesicles according to the embodiment of the present disclosure are vesicles with a lipid membrane structure, with a size between about 30 nm to 1000 nm. The vesicles encapsulate substances such as nucleic acids, peptides, proteins, and lipids. The PRP-derived extracellular vesicles express platelet-specific surface antigen CD41 as well as extracellular vesicle-specific surface antigens CD9, CD63 and Alix.

In the embodiment, the concentration of the PRP-derived extracellular vesicles in the composition may be 0.5 mg/mL to 2.5 mg/mL. In another embodiment, the concentration of the PRP-derived extracellular vesicles in the composition may be 0.5 mg/mL to 1 mg/mL. In other embodiment, the concentration of the PRP-derived extracellular vesicles in the composition may be 1.5 mg/mL to 2.5 mg/mL. In other embodiment, the concentration of the PRP-derived extracellular vesicles in the composition may be 1.25 mg/mL.

In the embodiment, the concentration of the PRP-derived extracellular vesicles in the composition may be 1% (v/v) to 5% (v/v). In another embodiment, the concentration of the PRP-derived extracellular vesicles in the composition may be 1% (v/v) to 2.5% (v/v). In other embodiment, the concentration of the PRP-derived extracellular vesicles in the composition may be 2.5% (v/v) to 5% (v/v). In other embodiment, the concentration of the PRP-derived extracellular vesicles in the composition may be 2.5% (v/v).

In the embodiment, the ratio of the PRP-derived extracellular vesicles to the mitochondria in the composition may be 1 mg:24 μg to 1 mg:320 μg. In another embodiment, the ratio of the PRP-derived extracellular vesicles to the mitochondria in the composition may be 1 mg:120 μg to 1 mg:320 μg. In other embodiment, the ratio of the PRP-derived extracellular vesicles to the mitochondria in the composition may be 1 mg:48 μg to 1 mg:128 μg. In other embodiment, the ratio of the PRP-derived extracellular vesicles to the mitochondria in the composition may be 1 mg:24 μg to 1 mg:64 μg.

In the embodiment, the ratio of the PRP-derived extracellular vesicles to the mitochondria in the composition may be 1 μL:2.56 μg to 1 μL:12.8 μg. In another embodiment, the ratio of the PRP-derived extracellular vesicles to the mitochondria in the composition may be 1 μL:2.56 μg to 1 μL:6.4 μg. In other embodiment, the ratio of the PRP-derived extracellular vesicles to the mitochondria in the composition may be 1 μL:6.4 μg to 1 μL:12.8 μg. In other embodiment, the ratio of the PRP-derived extracellular vesicles to the mitochondria in the composition may be 1 μL:6.4 μg.

According to another embodiment of the present disclosure, a use of mitochondria in manufacturing a composition for mitigating, repairing, ameliorating, or treating hearing loss is provided. The composition may be the above-described composition including mitochondria. The hearing loss may be ear cell damage, specifically damage of cochlear hair cells. The hearing loss may include conductive hearing loss, central hearing loss, sensorineural hearing loss or mixed hearing loss. The composition may improve the mitochondrial membrane potential of the ear cells, thereby improving the mitochondrial function of the ear cells. The composition may reduce the death of the ear cells, reduce reactive oxygen species produced by the ear cells, or improve the mitochondrial function of the ear cells, thereby mitigating, repairing, ameliorating, or treating hearing loss.

The materials used in the experiments are described as follows.

The mitochondria used in the embodiments of the present disclosure are taken from human adipose-derived mesenchymal stem cells (ADSCs), and the adipose-derived mesenchymal stem cells express surface makers CD73, CD90 and CD105, and no surface markers CD34 and CD45. The culture medium for the stem cells includes Keratinocyte SFM 1X solution (Gibco), bovine pituitary extract (BPE, Gibco), 10% (v/v) FBS (HyClone). First, ADSCs are cultured in a Petri dish to 1.5×10⁸ cells and then washed with Dulbecco's phosphate-buffered saline (DPBS). Next, DPBS is removed, trypsin for dissociating adherent cells from the Petri dish surfaces is added and reacted at 37° C. for 3 minutes, and the reaction is stopped by adding the culture medium for the stem cells. Next, ADSCs are washed down from the Petri dish, dispersed, and centrifuged at 600 g for 10 minutes, and the supernatant is removed. Next, the remained ADSCs and 80 mL of IBC-1 buffer (225 mM mannitol, 75 mM sucrose, 0.1 mM EDTA, and 30 mM Tris-HCl pH 7.4) are added to a homogenizer, and ADSCs are ground on ice by the homogenizer. Next, the ground ADSCs are centrifuged at 600 g and 4° C. for 5 minutes, and the supernatant is collected. Next, the supernatant is centrifuged at 10000 g and 4° C. for 10 minutes, and then the supernatant is removed. The mitochondria are extracted by using Mitochondria Isolation Kit, human (purchased from Miltenyi Biotec, Germany). Magnetic microbead antibody Anti-TOM22 is added to the resulting extract and reacted on ice for 1 hour, and then the mitochondria are purified by magnetic separation. The protein concentration is measured for the purified mitochondria and defined as the weight of the mitochondria.

The PRP-derived extracellular vesicles used in the embodiments of the present disclosure may be manufactured as follows. Whole blood is collected from the median cubital vein using a 19G butterfly needle, and the first 5 mL of the collected blood is discarded to collect about 20 mL of blood. The collected blood is placed in a 50 mL centrifuge tube containing 3.2% (v/v) of trisodium citrate. The blood is centrifuged at 2500 g for 15 minutes to collect about 5 to 6 mL of supernatant, which is platelet-rich plasma (PRP). 5 to 6 mL of the obtained PRP is mixed with phosphate-buffered saline (PBS) (without calcium and magnesium ions) at a ratio of 1:1, centrifuged at 10000 g and 4° C. for 120 minutes, and the supernatant is removed to obtain 30 to 70 mg of the PRP-derived extracellular vesicles. The obtained PRP-derived extracellular vesicles are resuspended with 1 mL of PBS to obtain about 50.05±17.66 mg/mL of the extracellular vesicles of PRP, hereinafter referred to as PRP-EVs. The PRP-derived extracellular vesicles express platelet-specific surface antigen CD41 as well as extracellular vesicle-specific surface antigens CD9, CD63 and Alix.

In the following experiments, the human cochlear hair cells (House Ear Institute-organ of Corti 1, HEI-OC1) are used to study hearing loss. The culture medium for the human cochlear hair cells may include DMEM (Dulbecco's Modified Eagle Medium) and 10% (v/v) fetal bovine serum (FBS), and the culture condition may be at 33° C. and 10% CO₂. The human cochlear hair cells are cultured until the culture dish is 90% full, then the culture medium is removed, and the cells are rinsed with phosphate buffered saline (PBS). Then, PBS is removed, and 0.25% of Trypsin is added to the culture dish and incubated at 33° C. for 5 minutes. After the incubation, the cells are centrifuged at 300 g for 5 minutes to remove the supernatant, and then fresh culture medium (DMEM containing 10% FBS) is added thereto to count the cells, and the cell subculture will be performed according to experimental requirements.

In the following experiment, Alamar blue kit (alamarBlue™ Cell Viability Reagent, purchased from Thermo Fisher) is used to analyze cell viability. Alamar blue, also known as resazurin, is a redox indicator, which is a nontoxic, cell-permeable, weakly fluorescent, and deep blue dye. Upon entering living cells, resazurin is reduced to resorufin, a compound that is pink and highly fluorescent, by coenzyme NADH. The cell viability or proliferation may be evaluated by detecting the absorbance or fluorescence of resorufin, such as at an excitation wavelength of OD530 and at an emission wavelength of OD595. The higher absorbance or fluorescence of resorufin indicates the more cells and the higher cell viability or proliferation. High viability or proliferation means that the cells are healthy and have a high proliferation ability.

In the following experiments, CM-H₂DCFDA (purchased from Invitrogen, C6827) is used to analyze reactive oxygen species (ROS) in cells. CM-H₂DCFDA may permeate the cell membrane and reacts with ROS inside the cells to form high-fluorescent products, and thus it is often used as an indicator for ROS.

In the following experiments, JC-1 dye (Invitrogen T3168, purchased from Fisher scientific) is used to analyze the mitochondrial membrane potential of cells. When the mitochondrial function of the cells is normal, the mitochondria are polarized, and the mitochondrial membrane potential is negatively charged. At this time, the positively charged JC-1 dye is accumulated on the mitochondrial membrane to form JC-1 aggregate and generate red fluorescence. When the mitochondrial function is damaged, the mitochondria are depolarized, and the mitochondrial membrane potential collapses. At this time, JC-1 dye does not form aggregate, and JC-1 monomers are distributed in the cells and generate green fluorescence. Therefore, a ratio of JC-1 monomer/JC-1 aggregate (hereinafter referred to as JC-1 ratio) may be obtained by fluorescent measurement and may be used as an indicator for evaluating the mitochondrial function. When JC-1 monomer/JC-1 aggregate is high, the mitochondrial membrane potential is poor, which indicates the mitochondrial function of the cells is poor.

Unless otherwise stated, the experimental values are presented as mean±standard deviation and are statistically analyzed by ANOVA test and Tukey post hoc test. In the following experiments, the peroxides, i.e. H₂O₂, are used as the damaging agent for the human cochlear hair cells.

### Experiment 1: Cytotoxicity of H₂O₂ to Human Cochlear Hair Cells

The human cochlear hair cells are cultured at a density of 25000 cells per well in 0.5 mL of the culture medium in a 24-well plate for 24 hours. Then, after the cells are cultured until the well is 80% full, the culture medium is removed, and the cells are rinsed with 0.5 mL PBS per well. Then, the rinsed PBS is removed, and a fresh DMEM without FBS (250 μL/well) is added. Then, H₂O₂ is added at a concentration of 0, 100, 250, 500, 1000, or 1500 μM in the well. After the cells are cultured with H₂O₂ for 1 hours, the cells are rinsed with 0.5 mL PBS per well. Then, the rinsed PBS is removed, and a fresh DMEM with 1% FBS (250 μL/well) is added to culture for 16 hours. After culture, the cell viability is analyzed by using Alamar blue kit.

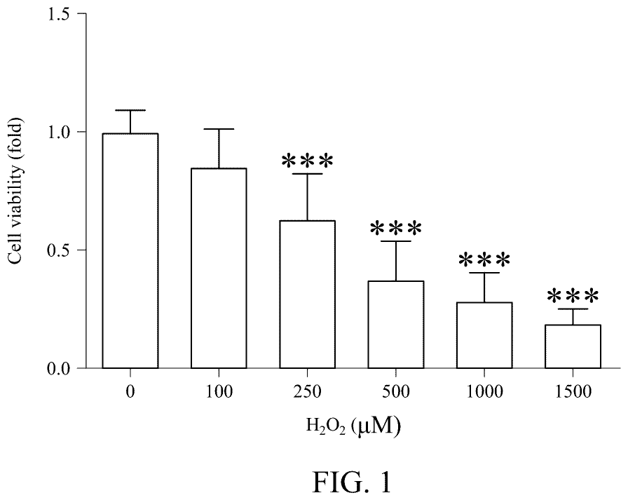

The experimental results are shown in Table 1 and FIG. 1. FIG. 1 shows the cell viability of the human cochlear hair cells treated with H₂O₂, relative to the control group. In FIG. 1, the control group is the cells without H₂O₂ (H₂O₂ is 0 μM), and the symbol “*” represents a statistically significant difference (*** indicates P<0.001) relative to the control group. From the experimental results, H₂O₂ causes damage to the human cochlear hair cells. In addition, the extent of the damage becomes more severe as the concentration of H₂O₂ increases.

TABLE 1

| H₂ O₂ (μM) | Cell viability (fold) |
|---|---|
| 0 | 1 ± 0.09 |
| 100 | 0.85 ± 0.16 |
| 250 | 0.63 ± 0.19 |
| 500 | 0.38 ± 0.16 |
| 1000 | 0.29 ± 0.12 |
| 1500 | 0.19 ± 0.06 |

### Experiment 2: Reactive Oxygen Species (ROS) Production of the Human Cochlear Hair Cells Induced by H₂O₂

The procedure in this experiment is generally the same as that in Experiment 1, and only the differences are described below. H₂O₂ is added at a concentration of 0, 250, or 500 μM in the well. The cells are cultured with H₂O₂ at 33° C. for 2 hours and then rinsed with 0.5 mL PBS per well. Then, the rinsed PBS is removed, and a fresh DMEM with 1% FBS and 10 μM CM-H₂DCFDA (250 μL/well) is added to react in the dark at 33° C. for 10 minutes. After reaction, the supernatant in the well is removed, and the cells are rinsed with 0.5 mL PBS per well. Then, the rinsed PBS is removed, and 250 μL per well of RIPA Lysis and Extraction Buffer (purchased from Thermo Scientific, 89900) is added to lysis the cells. The obtained solution is collected in a 1.5 mL tube and centrifuged at 300 g for 1 minute. 200 μL of the supernatant is loaded in a 96-well black plate, and the fluorescence is measured at OD485 (excitation) and OD530 (emission) to analyze the amount of ROS.

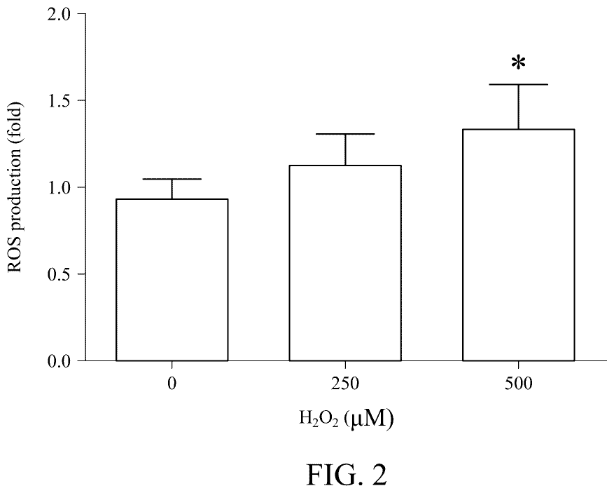

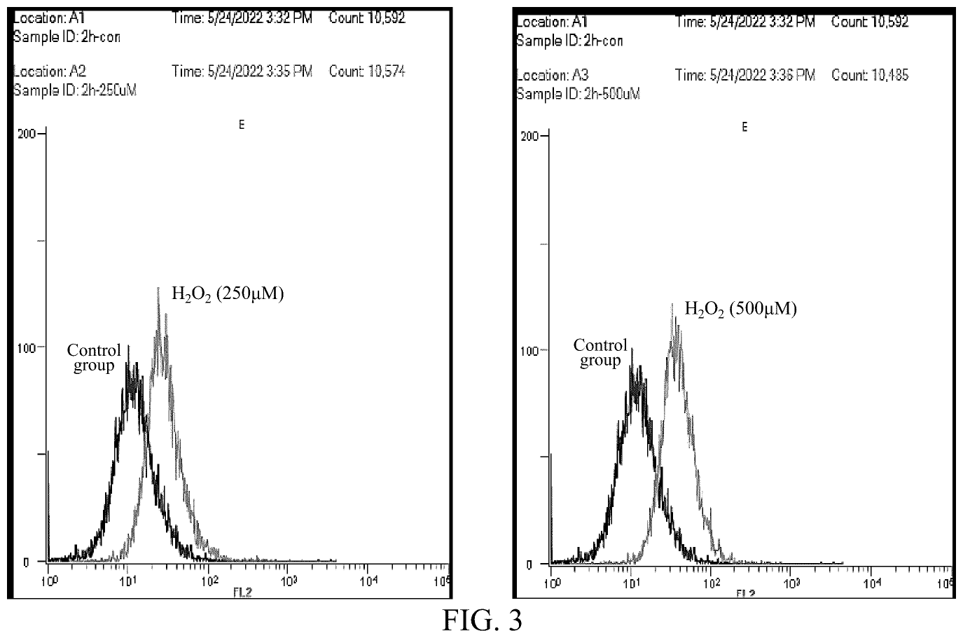

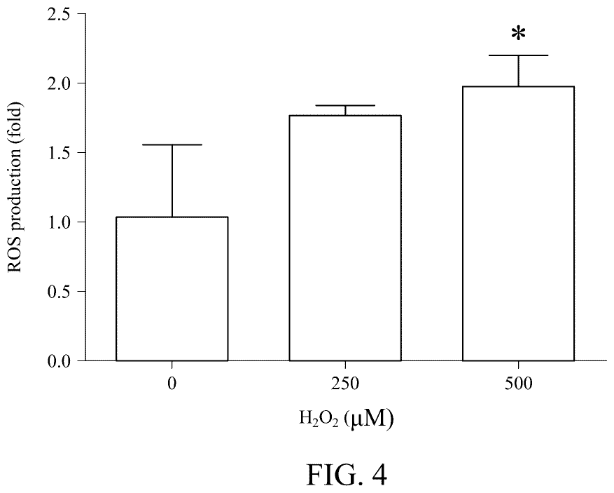

The experimental results are shown in Table 2 and FIGS. 2 to 4. FIG. 2 shows the ROS production of the human cochlear hair cells treated with H₂O₂, relative to the control group. FIG. 3 shows the flow cytometry analysis of the ROS production of the human cochlear hair cells treated with H₂O₂, relative to the control group. FIG. 4 shows the ROS production calculated from the flow cytometry analysis of FIG. 3, relative to the control group. In FIGS. 2 and 4, the control group is the cells without H₂O₂ (H₂O₂ is 0 μM), and the symbol “*” represents a statistically significant difference (* indicates P<0.05) relative to the control group. From the experimental results, H₂O₂ induces the human cochlear hair cells to produce ROS. In addition, the produced ROS increases as the concentration of H₂O₂ increases. The production and increase of ROS further cause oxidative damage to the human cochlear hair cells.

TABLE 2

| | | ROS production (fold) |
|---|---|---|
| | ROS production (fold) | (calculated from flow |
| H₂ O₂ (μM) | (CM-H₂ DCFDA) | cytometry analysis) |
| 0 | 1 ± 0.15 | 1.05 ± 0.51 |
| 250 | 1.14 ± 0.17 | 1.781 ± 0.06 |
| 500 | 1.34 ± 0.25 | 1.99 ± 0.21 |

### Experiment 3: Damage to the Mitochondria in the Human Cochlear Hair Cells Caused by H₂O₂

The procedure in this experiment is generally the same as that in Experiment 1, and only the differences are described below. H₂O₂ is added at a concentration of 0, 100, 250, 500, 1000, or 1500 μM in the well. After the cells are cultured with H₂O₂ at 33° C. for 1 hours, the supernatant in the well is removed, and the cells are rinsed with 0.5 mL PBS per well. Then, the rinsed PBS is removed, and a fresh DMEM with 1% FBS (500 μL/well) is added to culture for 16 hours. After culture, the supernatant in the well is removed, and the cells are rinsed with 0.5 mL PBS per well. The, the rinsed PBS is removed, and a fresh DMEM with 1% FBS and 5 μM JC-1 (250 μL/well) is added to react at 33° C. for 10 minutes. After reaction, the supernatant in the well is removed, and the cells are rinsed with 0.5 mL PBS per well twice. Then, a fresh DMEM with 10% FBS (250 μL/well) is added. The fluorescence of JC-1 aggregate is measured at OD520 (excitation) and OD590 (emission), and the fluorescence of JC-1 monomer is measured at OD490 (excitation) and OD530 (emission), thereby evaluating the mitochondrial membrane potential of the human cochlear hair cells.

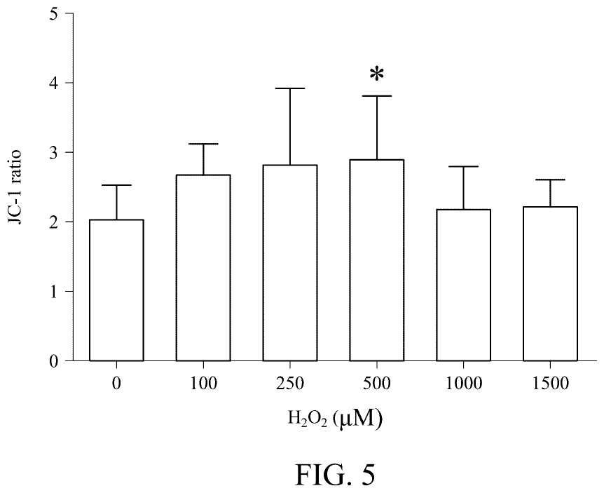

The experimental results are shown in Table 3 and FIG. 5. FIG. 5 shows the ratio of JC-1 monomer/JC-1 aggregate (JC-1 ratio) of the mitochondria in the human cochlear hair cells treated with H₂O₂. In FIG. 5, the control group is the cells without H₂O₂ (H₂O₂ is 0 μM), and the symbol “*” represents a statistically significant difference (* indicates P<0.05) relative to the control group. From the experimental results, H₂O₂ increases the ratio of JC-1 monomer/JC-1 aggregate (JC-1 ratio) of the mitochondria in the human cochlear hair cells, which indicates that the mitochondrial membrane is damaged, and the mitochondrial function is damaged. In addition, the extent of the damage to the mitochondria becomes more severe as the concentration of H₂O₂ increases. In addition, the high concentrations of H₂O₂ (1000 μM or 1500 μM) do not lead to a high JC-1 ratio, and a possible explanation may be that the high concentrations of H₂O₂ (1000 μM or 1500 μM) lead to cell death, causing both of JC-1 monomer and JC-1 aggregate are greatly decreased, thereby exhibiting a low JC-1 ratio.

TABLE 3

| H₂ O₂ (μM) | JC-1 ratio |
|---|---|
| 0 | 2.05 ± 0.47 |
| 100 | 2.70 ± 0.42 |
| 250 | 2.84 ± 1.09 |
| 500 | 2.92 ± 0.89 |
| 1000 | 2.20 ± 0.60 |
| 1500 | 2.24 ± 0.36 |

### Experiment 4: Mitochondria Reducing Cell Death of the Human Cochlear Hair Cells Caused by H₂O₂

The procedure in this experiment is generally the same as that in Experiment 1, and only the differences are described below. H₂O₂ is added at a concentration of 0 or 500 μM in the well. After the cells are cultured with H₂O₂ for 1 hours, the cells are rinsed with 0.5 mL PBS per well. Then, the rinsed PBS is removed, and a fresh DMEM with 1% FBS (250 μL/well) and the composition of each of the examples and comparative examples are added to culture for 16 hours. After cell culture, the cell viability is analyzed by using Alamar blue kit.

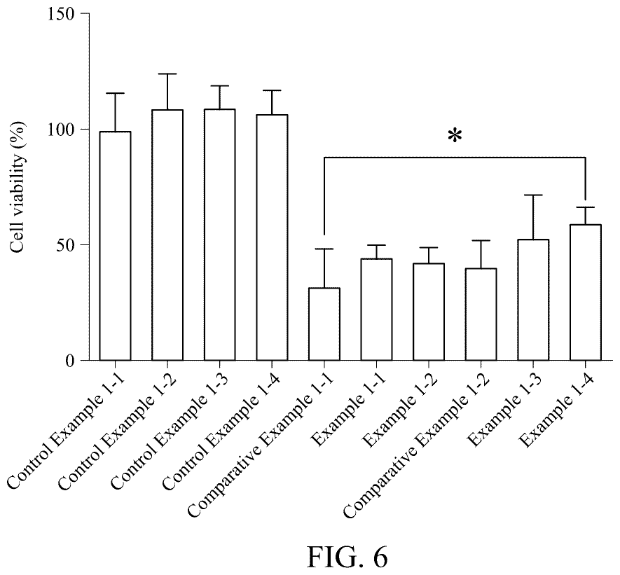

The experimental results are shown in Table 4 and FIG. 6. FIG. 6 shows the cell viability of the human cochlear hair cells treated with H₂O₂ and then treated with the compositions of the examples and the comparative examples, relative to the control group. In FIG. 6, the control group is the cells without H₂O₂, mitochondria, and PRP-EVs (Control Example 1-1), the symbol “*” represents a statistically significant difference (* indicates P<0.05). From Control Examples 1-1 to 1-4, in the case that the cells are not damaged, the addition of the mitochondria or PRP-EVs alone does not decrease the cell viability and can even slightly increase the cell viability, which indicates that the mitochondria and PRP-EVs have no cytotoxicity to the human cochlear hair cells, and it can be even said that the composition including the mitochondria or the composition including the PRP-EVs promotes the growth of the human cochlear hair cells. In addition, from the examples and the comparative examples, in the case that the cells are damaged, the addition of the mitochondria can increase the cell viability (Examples 1-1 and 1-2), which indicates that the addition of the mitochondria contributes to mitigating, repairing, ameliorating, or treating the damage to the human cochlear hair cells caused by H₂O₂, further reducing the death of the human cochlear hair cells caused by H₂O₂. Further, from the examples and the comparative examples, in the case that the cells are damaged, the addition of the composition including the mitochondria and PRP-EVs can further increase the cell viability (Examples 1-3 and 1-4) and have a statistically significant difference, which indicates the addition of the composition including the mitochondria and PRP-EVs exhibits a synergistic effect in mitigating, repairing, ameliorating, or treating the damage to the human cochlear hair cells, and can significantly reduce the death of the human cochlear hair cells caused by H₂O₂.

TABLE 4

| | H₂ O₂ | Mitochondria | | PRP-EVs | Cell viability |
|---|---|---|---|---|---|
| Group | μM | μg | μg/mL | v/v % | % |
| Control | — | — | — | — | 100 ± 15.6 |
| Example 1-1 | | | | | |
| Control | | 15 | 60 | — | 109.4 ± 14.8 |
| Example 1-2 | | | | | |
| Control | | 40 | 160 | — | 109.6 ± 9.3 |
| Example 1-3 | | | | | |
| Control | | — | — | 2.5 | 107.2 ± 9.6 |
| Example 1-4 | | | | | |
| Comparative | 500 | — | — | — | 32.1 ± 16.2 |
| Example 1-1 | | | | | |
| Example 1-1 | | 15 | 60 | — | 44.8 ± 5.2 |
| Example 1-2 | | 40 | 160 | — | 42.9 ± 6.0 |
| Comparative | | — | — | 2.5 | 40.6 ± 11.4 |
| Example 1-2 | | | | | |
| Example 1-3 | | 15 | 60 | 2.5 | 53.2 ± 18.5 |
| Example 1-4 | | 40 | 160 | 2.5 | 59.7 ± 6.9 |

### Experiment 5: Mitochondria Reducing ROS Production of the Human Cochlear Hair Cells Induced by H₂O₂

The procedure in this experiment is generally the same as that in Experiment 1, and only the differences are described below. H₂O₂ is added at a concentration of 0 or 500 M in the well. After the cells are cultured with H₂O₂ for 2 hours, the cells are rinsed with 0.5 mL PBS per well. Then, the rinsed PBS is removed, and a fresh DMEM with 1% FBS (250 μL/well) and the composition of each of the examples and comparative examples are added to culture for 16 hours. After cell culture, the cells are rinsed with 0.5 mL PBS per well. Then, the rinsed PBS is removed, and a fresh DMEM with 1% FBS and 10 μM CM-H₂DCFDA (250 μL/well) is added to react in the dark at 33° C. for 10 minutes. After reaction, the supernatant in the well is removed, and the cells are rinsed with 0.5 mL PBS per well. Then, the rinsed PBS is removed, and 250 μL per well of RIPA Lysis and Extraction Buffer (purchased from Thermo Scientific, 89900) is added to lysis the cells. The obtained solution is collected in a 1.5 mL tube and centrifuged at 300 g for 1 minute. 200 μL of the supernatant is loaded in a 96-well black plate, and the fluorescence is measured at OD485 (excitation) and OD530 (emission) to analyze the amount of ROS.

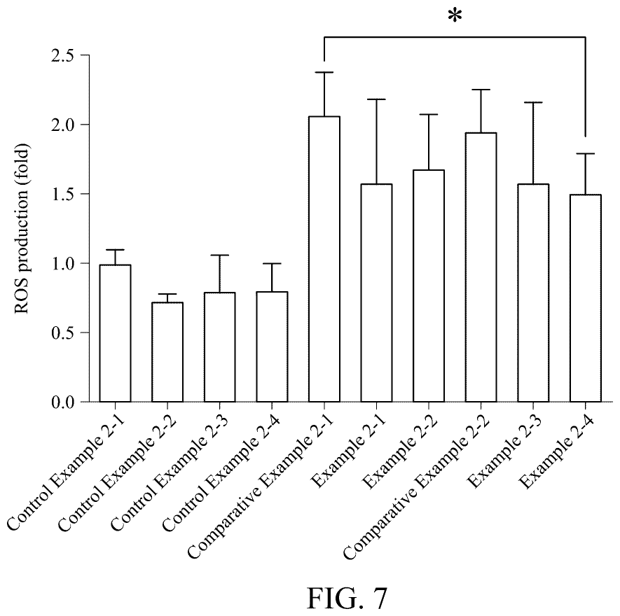

The experimental results are shown in Table 5 and FIG. 7. FIG. 7 shows the ROS production of the human cochlear hair cells treated with H₂O₂ and then treated with the compositions of the examples and the comparative examples, relative to the control group. In FIG. 7, the control group is the cells without H₂O₂, mitochondria, and PRP-EVs (Control Example 2-1), the symbol “*” represents a statistically significant difference (* indicates P<0.05). From Control Examples 2-1 to 2-4, in the case that the cells are not damaged, the addition of the mitochondria or PRP-EVs alone does not affect ROS production and can even slightly decrease the ROS production, which indicates that the mitochondria and PRP-EVs does not induce the human cochlear hair cells to produce ROS, and it can be even said that the composition including the mitochondria or the composition including the PRP-EVs facilitates the reduction of the ROS production of the human cochlear hair cells. In addition, from the examples and the comparative examples, in the case that the cells are damaged, the addition of the mitochondria can reduce ROS production (Examples 2-1 and 2-2), which indicates that the addition of the mitochondria contributes to mitigating, repairing, ameliorating, or treating the damage to the human cochlear hair cells caused by H₂O₂, and reduces the damage further caused by ROS. Further, from the examples and the comparative examples, in the case that the cells are damaged, the addition of the composition including the mitochondria and PRP-EVs can further reduce ROS production compared to the addition of an equivalent amount of the mitochondria (Examples 2-3 and 2-4) and have a statistically significant difference, which indicates the addition of the composition including the mitochondria and PRP-EVs exhibits a synergistic effect in mitigating, repairing, ameliorating, or treating the damage to the human cochlear hair cells, and can significantly reduce the damage further caused by ROS.

TABLE 5

| | | | | | ROS |
|---|---|---|---|---|---|
| | H2O2 | Mitochondria | | PRP-EVs | production |
| Group | μM | μg | μg/mL | v/v % | fold |
| Control | — | — | — | — | 1 ± 0.09 |
| Example 2-1 | | | | | |
| Control | | 15 | 60 | — | 0.73 ± 0.05 |
| Example 2-2 | | | | | |
| Control | | 40 | 160 | — | 0.80 ± 0.25 |
| Example 2-3 | | | | | |
| Control | | — | — | 2.5 | 0.80 ± 0.19 |
| Example 2-4 | | | | | |
| Comparative | 500 | — | — | — | 2.07 ± 0.30 |
| Example 2-1 | | | | | |
| Example 2-1 | | 15 | 60 | — | 1.58 ± 0.59 |
| Example 2-2 | | 40 | 160 | — | 1.68 ± 0.39 |
| Comparative | | — | — | 2.5 | 1.95 ± 0.29 |
| Example 2-2 | | | | | |
| Example 2-3 | | 15 | 60 | 2.5 | 1.58 ± 0.58 |
| Example 2-4 | | 40 | 160 | 2.5 | 1.51 ± 0.28 |

### Experiment 6: Mitochondria Reducing the Damage to the Mitochondria in the Human Cochlear Hair Cells Caused by H₂O₂

The procedure in this experiment is generally the same as that in Experiment 1, and only the differences are described below. H₂O₂ is added at a concentration of 0 or 500 μM in the well. After the cells are cultured with H₂O₂ at 33° C. for 1 hours, the supernatant in the well is removed, and the cells are rinsed with 0.5 mL PBS per well. Then, the rinsed PBS is removed, and a fresh DMEM with 1% FBS (550 μL/well) and the composition of each of the examples and comparative examples are added to culture for 16 hours. After cell culture, the supernatant in the well is removed, and the cells are rinsed with 0.5 mL PBS per well. Then, the rinsed PBS is removed, and a fresh DMEM with 1% FBS and 5 μM JC-1 (250 μL/well) is added to react at 33° C. for 10 minutes. After reaction, the supernatant in the well is removed, and the cells are rinsed with 0.5 mL PBS per well twice. Then, a fresh DMEM with 1% FBS (250 μL/well) is added. The fluorescence of JC-1 aggregate is measured at OD520 (excitation) and OD590 (emission), and the fluorescence of JC-1 monomer is measured at OD490 (excitation) and OD530 (emission), thereby evaluating the mitochondrial membrane potential of the human cochlear hair cells.

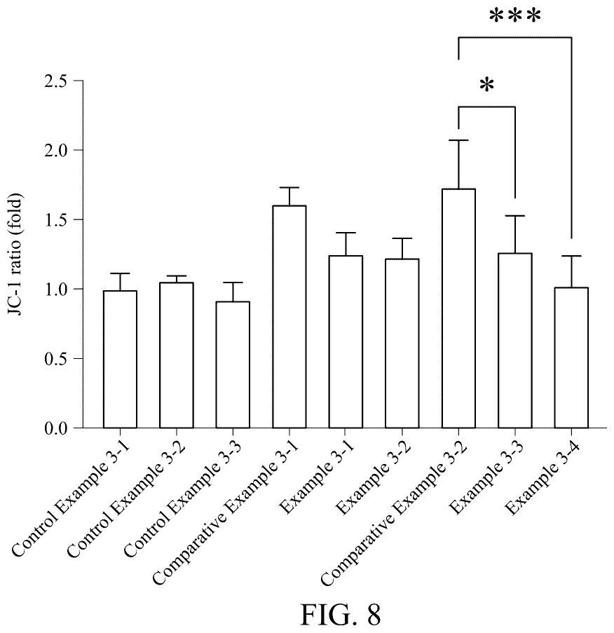

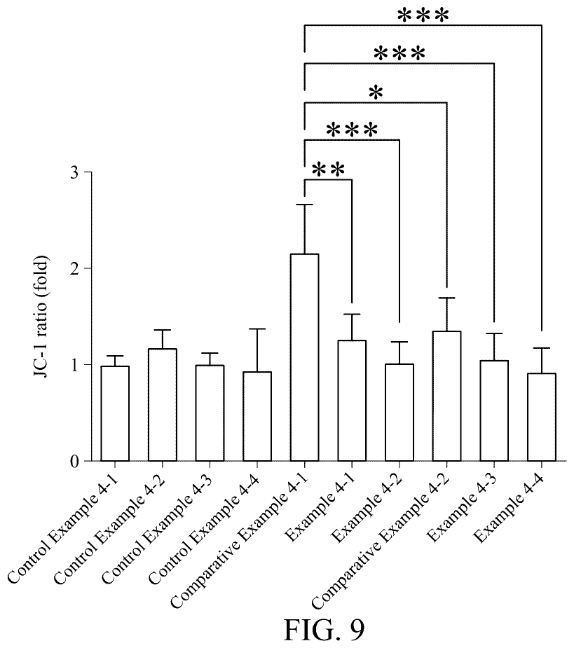

The experimental results are shown in Tables 6 and 7 and FIGS. 8 and 9. Tables 6 and 7 and FIGS. 8 and 9 show the experimental results from different batches, respectively. FIGS. 8 and 9 show the ratio of JC-1 monomer/JC-1 aggregate (JC-1 ratio) of the mitochondria in the human cochlear hair cells treated with H₂O₂ and then treated with the compositions of the examples and the comparative examples, relative to the control group. In FIGS. 8 and 9, the control group is the cells without H₂O₂, mitochondria, and PRP-EVs (Control Examples 3-1 and 4-1), the symbol “*” represents a statistically significant difference (*indicates P<0.05, ** indicates P<0.01, and *** indicates P<0.001) relative to the comparative example (Comparative Examples 3-2 and 4-1). From Control Examples 3-1 to 3-3 and 4-1 to 4-4, in the case that the cells are not damaged, the addition of the mitochondria or PRP-EVs alone does not affect the ratio of JC-1 monomer/JC-1 aggregate (JC-1 ratio) of the mitochondria in the human cochlear hair cells, which indicates that the mitochondria and PRP-EVs have no adverse effect on the mitochondrial function of the human cochlear hair cells. In addition, from the examples and the comparative examples, in the case that the cells are damaged, the addition of the mitochondria can decrease the JC-1 ratio (Examples 3-1 to 3-4 and 4-1 to 4-2), which indicates that the damaged mitochondrial membrane to the human cochlear hair cells is improved, and further indicates that the addition of the mitochondria can reduce the damage to the mitochondria in the human cochlear hair cells caused by H₂O₂ and improve the mitochondrial function of the human cochlear hair cells. Further, from Examples 4-3 and 4-4, in the case that the cells are damaged, the addition of the composition including the mitochondria and PRP-EVs can further decrease the JC-1 ratio (Examples 4-3 and 4-4) and have a statistically significant difference, which indicates the addition of the composition including the mitochondria and PRP-EVs exhibits a synergistic effect in mitigating, repairing, ameliorating, or treating the damage to the human cochlear hair cells, and can further improve the mitochondrial function of the human cochlear hair cells.

TABLE 6

| | | |
|---|---|---|
| H₂ O₂ | Mitochondria | JC-1 ratio |

| Group | μM | μg | μg/mL | fold |
|---|---|---|---|---|
| Control | — | — | — | 1 ± 0.11 |
| Example 3-1 | | | | |
| Control | | 15 | 60 | 1.06 ± 0.03 |
| Example 3-2 | | | | |
| Control | | 40 | 160 | 0.93 ± 0.12 |
| Example 3-3 | | | | |
| Comparative | 250 | — | — | 1.61 ± 0.12 |
| Example 3-1 | | | | |
| Example 3-1 | | 15 | 60 | 1.25 ± 0.15 |
| Example 3-2 | | 40 | 160 | 1.23 ± 0.13 |
| Comparative | 500 | — | — | 1.73 ± 0.33 |
| Example 3-2 | | | | |
| Example 3-3 | | 15 | 60 | 1.27 ± 0.25 |
| Example 3-4 | | 40 | 160 | 1.03 ± 0.21 |

TABLE 7

| | H₂ O₂ | Mitochondria | | PRP-EVs | JC-1 ratio |
|---|---|---|---|---|---|
| Group | μM | μg | μg/mL | v/v % | fold |
| Control | — | — | — | — | 1 ± 0.09 |
| Example 4-1 | | | | | |
| Control | | 15 | 60 | — | 1.18 ± 0.19 |
| Example 4-2 | | | | | |
| Control | | 40 | 160 | — | 1.01 ± 0.11 |
| Example 4-3 | | | | | |
| Control | | — | — | 2.5 | 0.94 ± 0.43 |
| Example 4-4 | | | | | |
| Comparative | 500 | — | — | — | 2.17 ± 0.49 |
| Example 4-1 | | | | | |
| Example 4-1 | | 15 | 60 | — | 1.27 ± 0.25 |
| Example 4-2 | | 40 | 160 | — | 1.03 ± 0.21 |
| Comparative | | — | — | 2.5 | 1.36 ± 0.33 |
| Example 4-2 | | | | | |
| Example 4-3 | | 15 | 60 | 2.5 | 1.06 ± 0.26 |
| Example 4-4 | | 40 | 160 | 2.5 | 0.93 ± 0.24 |

According to the above experiments and the embodiments of the present disclosure, the composition including mitochondria may reduce the damage caused by peroxides to ear cells, thereby reducing the death of ear cells. Also, the composition including mitochondria may reduce the production of reactive oxygen species (ROS) in ear cells induced by peroxides, thereby reducing the further damage caused by the reactive oxygen species to ear cells. In addition, the composition including mitochondria may reduce the damage caused by peroxides to the mitochondria in ear cells, thereby improving the mitochondrial function of ear cells. Further, the composition including mitochondria and extracellular vesicles exhibits synergistic effects in mitigating, repairing, ameliorating, or treating the damage to ear cells, significantly reduces the death of ear cells caused by peroxides, further reduces the production of reactive oxygen species and the associated damage, and further improves the mitochondrial function of ear cells. Therefore, the composition of the embodiments of the present disclosure may achieve the purposes of mitigating, repairing, ameliorating, or treating hearing loss, and may be expected to be a composition or medicament that is able to mitigate, repair, ameliorate or treat hearing loss while having both safety and effectiveness.

What is claimed is:

1. A use of mitochondria in manufacturing a composition for hearing loss mitigation.

2. The use of claim 1, wherein the hearing loss is due to ear cell damage.

3. The use of claim 2, the ear cells comprise cochlear hair cells, cochlear nerve cells, spiral ganglion neurons, or vestibular nerve cells.

4. The use of claim 1, wherein the hearing loss comprises conductive hearing loss, central hearing loss, sensorineural hearing loss, or mixed hearing loss.

5. The use of claim 1, wherein the hearing loss mitigation comprises reducing reactive oxygen species produced by ear cells.

6. The use of claim 1, wherein the hearing loss mitigation comprises improving the mitochondrial function of ear cells.

7. The use of claim 1, wherein the hearing loss mitigation comprises improving the mitochondrial membrane potential of ear cells.

8. The use of claim 1, wherein an effective dose of the mitochondria in the composition is at least 15 μg.

9. The use of claim 1, wherein the composition further comprises extracellular vesicles.

10. The use of claim 9, wherein the extracellular vesicles are derived from platelet-rich plasma.

11. The use of claim 10, wherein the hearing loss mitigation comprises reducing the death of ear cells.

12. A composition, comprising mitochondria and a biocompatible carrier.

13. The composition of claim 12, wherein an effective dose of the mitochondria in the composition is at least 15 μg.

14. The composition of claim 12, further comprising extracellular vesicles.

15. The composition of claim 14, wherein the extracellular vesicles is derived from platelet-rich plasma.
